(19) United States
(12) Patent Application Publication
(10) Pub. No.: US 2003/0152540 A1
(43) Pub. Date: Aug. 14, 2003

(54) Rinse-off skin conditioning compositions

(76) Inventors: Christopher Dean Putman, West Chester, OH (US); Edward Dewey Smith III, Mason, OH (US); Cheyne Pohlman Thomas, Highland Heights, KY (US); Karl Shiqing Wei, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/358,803

(22) Filed: Feb. 5, 2003

Related U.S. Application Data
(60) Provisional application No. 60/355,327, filed on Feb. 8, 2002.

(51) Int. Cl. A61K 7/06; A61K 7/00

(52) U.S. Cl. 424/70.1; 424/74

(57) ABSTRACT

Skin conditioning compositions, and corresponding methods of application, wherein the compositions comprise a lipophilic carrier, solid particulates and preferably a skin benefit agent; and wherein the compositions have a Deposition Efficiency of at least about 30%, provide skin conditioning benefits. Preferred embodiments are further defined by selected lipophilic carrier rheologies, defined solid particulates for improved skin feel, and selected skin benefit agents for use in the composition. These compositions and corresponding methods provide improved cosmetics, skin feel, and/or skin active efficacy.

### CROSS REFERENCE TO RELATED APPLICATIONS

This application claims the benefit of U.S. Provisional Application No. 60/355,327, filed Feb. 8, 2002.

### FIELD OF THE INVENTION

The present invention relates to rinse-off, skin conditioning compositions comprising a lipophillic material and solid particulates.

### BACKGROUND OF THE INVENTION

Skin cleansing compositions that provide skin conditioning benefits are known. Many of these compositions are aqueous systems comprising an emulsified conditioning oil or other similar material in combination with a lathering surfactant. Although it is convenient to use a single composition or product that provides both conditioning and cleansing benefits, it is often difficult to formulate a physically stable emulsion that contains an effective combination of a cleansing surfactant and a skin conditioning material.

It is additionally a tremendous challenge to deposit effective amounts of skin conditioning ingredients on skin via a rinse-off skin conditioning composition. While not to be bound in theory, it is believed that the conditioning agents are easily emulsified by the surfactant present in most body wash compositions. Therefore, the conditioning agents are rinsed away during personal cleansing process. Although attempts have been made to formulate two-in-on body wash products that not only cleanse the skin, but additionally deliver skin moisturization, they don't generally deposit sufficient amount of skin conditioning ingredients to deliver the same level of skin moisturization as a leave-on lotion.

One way to address the deposition deficiency is the development of rinse-off skin conditioner composition. U.S. Pat. Nos. 5,578,299 and 5,888,492, Starch, and 5,928,632, Reusch, disclose rinse-off skin conditioner compositions that may be applied when showering and then rinsed away. However, deposition is limited to about 3-25 percent by weight because amounts substantially higher than 25 percent by weight of the formula, as applied, are considered aesthetically undesirable by users. This does not provide adequate conditioning and leaves consumers with an unmet need.

Accordingly, the need remains for a rinse-off, skin-conditioning composition that can provide improved conditioning benefits to human skin. Additionally, there remains a need for a rinse-off, skin-conditioning composition which exhibits pleasing tactile properties and increased deposition of skin conditioning and/or skin benefit agents.

Applicants have discovered a way to increase deposition of skin conditioning agents on the skin with a rinse-off skin conditioner while maintaining aesthetics pleasing to most consumers. These compositions provide improved cosmetics and skin feel during or after application, especially reduced greasy skin feel.

The present invention also provides rinse-off, skin-conditioning compositions further comprising skin benefit agents. These compositions provide improved cosmetics and skin feel during and/or after application, and are especially useful in providing improved deposition or effectiveness of selected skin benefit agents to the desired area of the skin.

The present invention further provides a method of conditioning the skin using the described compositions.

### SUMMARY OF THE INVENTION

The present invention meets the aforementioned needs by providing a rinse-off, skin-conditioning compositions comprising a lipophilic carrier; and solid particulates, wherein the composition has a Deposition Efficiency (DE) of at least about 30% wherein DE=[W_{after}−W₀]/[W_{before}−W₀]×100%].

The present invention is further related to skin conditioning compositions comprising

at least about 10% by weight of a lipophilic carrier; and from about 1.0% to about 90% by weight of solid particulates, wherein the composition has a Deposition Efficiency (DE) of at least about 30% wherein DE=[W_{after}−W₀]/[W_{before}−W₀]×100%.

The present invention is further related to skin conditioning compositions wherein the lipophilic carrier has a Vaughn Solubility Parameter of from about 5 to about 10, preferably from about 6 to about 10, more preferably from about 6 to about 9.

The present invention is further related to skin conditioning compositions wherein the lipophilic carrier has a Consistency value of from about 1 poise to about 2,000 poise, preferably from about 10 to about 1,000 poise, more preferably from about 50 to about 1,000 poise.

The present invention is further related to skin conditioning compositions wherein the lipophilic carrier has a Shear Index value of from about 0.1 to about 0.8, preferably from about 0.1 to about 0.5, more preferably from about 0.20 to about 0.4.

The present invention further relates to a method of conditioning the skin comprising the steps of applying a skin conditioning composition as described above and removing the composition once applied by means selected from by rinsing, wiping and mixtures thereof.

The present invention also relates to skin conditioning compositions and methods for conditioning the skin with said compositions wherein the compositions further comprise a skin benefit agent. The skin benefit agent may be selected from the group consisting of desquamation actives, anti-acne actives, anti-wrinkle and anti-atrophy actives, anti-oxidants and radical scavengers, chelators, flavonoids, anti-inflammatory agents, anti-cellulite agents, topical anesthetics, tanning actives, skin lightening agents, skin soothing and skin healing actives, antimicrobial actives, and combinations thereof.

### DETAILED DESCRIPTION

The specific embodiments of the present invention will be described in detail below.

The term “anhydrous” as used herein, unless otherwise specified, refers to those compositions or materials containing less than about 10%, more preferably less than about 5%, even more preferably less than about 3%, even more preferably zero percent, by weight of water.

The term “volatile” as used herein, unless otherwise specified, refers to those materials having an average boiling point at one (1) atmosphere of pressure (atm) of less than about 250° C., more typically less than about 235° C. at one (1) atm.

The term “ambient conditions” as used herein, unless otherwise specified, refers to surrounding conditions at one (1) atmosphere of pressure, 50% relative humidity, and 25° C.

The term “skin conditioning composition” as used herein, unless otherwise specified, refers to the compositions of the present invention, wherein the compositions are intended for topical application to the hair or skin.

The Vaughan Solubility Parameter (VSP) as used herein is a parameter used to define the solubility of lipophilic materials. Vaughan Solubility parameters are well known in the various chemical and formulation arts and typically have a range of from 5 to 25.

The term “Consistency” or “k” as used herein is a measure of lipid viscosity and is used in combination with Shear index, to define viscosity for materials whose viscosity is a function of shear. The measurements are made at 35° C. and the units are poise (equal to 100 cps).

The term “Shear index” or “n” as used herein is a measure of lipid viscosity and is used in combination with Consistency, to define viscosity for materials whose viscosity is a function of shear. The measurements are made at 35° C. and the units are dimensionless.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

The skin conditioning compositions and methods of the present invention can comprise, consist of, or consist essentially of, the essential elements and limitations of the invention described herein, as well as any additional or optional ingredients, components, or limitations described herein or otherwise useful in skin conditioning compositions intended for topical application to the hair or skin.

### Product Form

The skin conditioning compositions of the present invention are liquid or semi-liquid compositions intended for topical application to the hair or skin. All of the product forms contemplated for purposes of defining the compositions and methods of the present invention are rinse-off formulations, by which is meant the product is applied topically to the hair or skin and then subsequently and immediately (i.e., within minutes) rinsed away with water, or otherwise wiped off using a substrate or other suitable removal means.

The skin conditioning compositions of the present invention comprise a selectively defined lipophilic carrier, and preferably a skin benefit agent suitable for application to the skin. The skin conditioning compositions are preferably single or multi-phase liquids within which the skin benefit agent is suspended, dispersed or otherwise dissolved in the selectively defined lipophilic carrier.

Suitable skin benefit agents for use herein include any known or otherwise effective skin benefit agent suitable for application to the skin, and which is otherwise compatible with the other essential ingredients in the skin conditioning compositions. Preferred are those skin benefit agents that provide chronic skin benefits.

The skin conditioning compositions also comprise at least about 10% by weight of one or more lipophilic carriers having a defined Vaugh Solubility Parameter. The lipophilic carriers for use in the skin conditioning compositions are preferably selected among those having defined rheological properties as described hereinafter.

The lipophilic carriers and skin benefit agents suitable for use in the skin conditioning compositions of the composition are described hereinafter in detail.

Lipophilic Carrier

The lipophilic carrier or carriers for use in the skin conditioning compositions represent from at least about 10% to about 99% by weight of the skin conditioning compositions. The lipophilic carriers are preferably selected among those having defined solubility and rheological properties as described hereinafter, including selected Vaughan Solubility Parameter Values (VSP), Consistency (k) and Shear Index (n). These preferred rheological properties are especially useful in providing the skin conditioning compositions with improved performance, including improved cosmetic and active efficacy benefits.

A) Vaughan Solubility Parameter Value (VSP)

The lipophilic carrier for use in the skin conditioning compositions preferably has a Vaughan Solubility Parameter (VSP) of from about 5 to about 10, preferably from about 6 to about 10, more preferably from about 6 to about 9. These solubility parameters are well known in the formulation arts, and are defined by Vaughan in Cosmetics and Toiletries, Vol. 103, p47-69, October 1988.

Non-limiting examples of lipophilic carriers having VSP values ranging from about 5 to about 10 include the following:

VAUGHAN SOLUBILITY PARAMETERS*

| | |
|---|---|
| Cyclomethicone | 5.92 |
| Squalene | 6.03 |
| Petrolatum | 7.33 |
| Isopropyl Palmitate | 7.78 |
| Isopropyl Myristate | 8.02 |
| Castor Oil | 8.90 |
| Cholesterol | 9.55 |

*As reported in Solubility, Effects in Product, Package, Penetration and Preservation, C. D. Vaughan, Cosmetics and Toiletries, Vol. 103, October 1988.

B) Rheology

The lipophilic carrier or carriers for use in the skin conditioning compositions have a preferred rheology profile as defined by Consistency (k) and Shear Index (n). Preferred Consistency and Shear Index ranges of are as follows:

| | k | n |
|---|---|---|
| Range | poise (1/sec)n-1 | (dimensionless) |
| Most preferred | 50-1000 | 0.20-0.4 |
| More Preferred | 10-1000 | 0.1-0.5 |
| Preferred | 1-2000 | 0.1-0.8 |

The lipophilic carriers can be characterized by Consistency (k) and Shear Index (n) values as defined by the above-described ranges, wherein these defined ranges are selected to provide enhanced deposition and reduced stickiness during and after application of the personal cleaning composition on hair or skin.

The Shear index (n) and Consistency (k) values are well known and accepted industry standards for reporting the viscosity profile of materials having a viscosity that is a function of an applied shear rate. The viscosity (μ) for any material can be characterized by the relationship or equation

[μ=σ/γ′]

wherein σ is shear stress and γ′ is shear rate, so that the viscosity for any material can be measured by either applying a shear rate and measuring the resultant shear stress or vice versa.

The Carrimed CSL 100 Controlled Stress Rheometer is used to determine Shear Index, n, and Consistency, k, for the lipophilic carriers herein. The determination is performed at 35° C. with the 4 cm 2° cone measuring system typically set with a 51 micron gap and is performed via the programmed application of a shear stress (typically from about 0.06 dynes/sq. cm to about 5,000, dynes/sq. cm) over time. If this stress results in a deformation of the sample, i.e. strain of the measuring geometry of at least 10-4 rad/sec, then this rate of strain is reported as a shear rate. These data are used to create a viscosity (μ) versus shear rate (γ′) flow curve for the lipophilic carrier material. This flow curve can then be modeled in order to provide a mathematical expression that describes the material's behavior within specific limits of shear stress and shear rate. These results were fitted with the following well-accepted power law model (see for instance: Chemical Engineering, by Coulson and Richardson, Pergamon, 1982 or Transport Phenomena by Bird, Stewart and Lightfoot, Wiley, 1960):

[μ=k(γ′)ⁿ⁻¹]

The Carrimed CSL 100 Controlled Stress Rheometer is used to perform oscillatory tests at 35° C. with the 4 cm 2° cone measuring system typically set with a 51 micron gap. The oscillatory tests at 35° C. are carried out in 2 steps. The first step is a stress amplitude sweep at the expected starting and ending frequencies for the frequency sweep. These tests allow a determination to be made as to whether or not the test conditions are within the linear viscoelastic region for the test material over the anticipated frequency range. The linear viscoelastic region is a region where there is a linear relationship between stress and strain. The second step is a frequency sweep made at a stress level within that linear viscoelastic region. The frequency sweep allows the test material's viscoelastic behavior to be measured. The oscillatory test on a controlled stress rheometer is performed by applying a stress in an oscillatory manner and measuring the resulting oscillatory strain response and the phase shift between the applied stress wave form and the resulting strain wave form in the test material. The resulting complex modulus is expressed as a combination of the material's elastic (G′) and viscous (G″) components. The elastic modulus G′ is a measure of a materials ability to store recoverable energy. This energy storage can be the result of the ability of a complex polymer, structural network, or a combination of these to recover stored energy after a deformation. The viscous or loss modulus G″ is a measure of the unrecoverable energy, which has been lost due to viscous flow.

The lipophilic carriers suitable for use herein can include a variety of hydrocarbons oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin and its derivatives, wax esters, beeswax derivatives, sterols and phospholipids, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, and combinations thereof.

Non-limiting examples of silicone oils suitable for use as lipophilic carriers herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.), which description is incorporated herein by reference.

Non-limiting examples of diglycerides and triglycerides suitable for use as lipophilic carriers herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof.

Non-limiting examples of acetoglyceride esters suitable for use as lipophilic carriers herein include acetylated monoglycerides.

Non-limiting examples of alkyl esters suitable for use as lipophilic carriers herein include isopropyl esters of fatty acids and long chain esters of long chain fatty acids, e.g. cetyl ricinoleate, non-limiting examples of which incloude isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as lipophilic carriers herein include oleyl myristate, oleyl stearate, oleyl oleate, and combinations thereof.

Non-limiting examples of lanolin and lanolin derivatives suitable for use as lipophilic carriers herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Still other suitable lipophilic carriers include milk triglycerides (e.g., hydroxylated milk glyceride) and polyol fatty acid polyesters.

Still other suitable lipophilic carries include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

The skin conditioning compositions of the present invention preferably comprises one or more lipophilic carriers, wherein at least 10% by weight of the lipophilic carriers are selected from petrolatum, mineral oil, sunflower seed oil, micro-crystalline waxes, paraffins, ozokerite, polyethylene, polybutene, polydecene and perhydrosqualene dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, maleated soy bean oil, safflower oil, cotton seed oil, corn oil, walnut oil, peanut oil, olive oil, cod liver oil, almond oil, avocado oil, palm oil and sesame oil, and combinations thereof. More preferably, at least about 50% by weight of the liophillic carriers are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the lipid is preferably selected from: isopropyl palmitate, cetyl riconoleate, octyl isononanoate, octyl palmitate, isocetyl stearate, hydroxylated milk glyceride and combinations thereof.

The preferred rheological properties as described herein are believed to provide improved deposition of the defined lipophilic materials.

Solid Particulates

The skin conditioning compositions of the present invention additionally comprise a solid particulate preferably having an average particle diameter of from about 1 μm to about 100 μm, more preferably from about 5 μm to about 40 μm, at concentrations ranging from about 1% to about 90%, more preferably from about 5% to about 40%, even more preferably from about 10% to about 40%, most preferably from about 10% to about 30%, by weight of the skin conditioning compositions.

Preferably, the solid particulates for use in the skin conditioning compositions are non-structuring particulates. In this context, the term non-structuring refers to those solid particulates that do no provide a substantial network structure to a composition, and therefore when formulated into the skin conditioning compositions of the present invention do not increase the phase viscosity by more than a factor of 3, preferably no more than by a factor of 2, as measured by a Brookfield DV-II+ viscometer at 1 rpm at 25° C. These non-structuring particulates therefore specifically exclude solid particulates that are commonly used as structurants or gellant materials, except that such materials can be used herein as non-structuring particulates provided that they are used at a concentration and under circumstances that do not result in an increase in phase viscosity as described above, or are otherwise used in the composition for any purpose other than to increase viscosity or structure of the skin conditioning compositions.

It has been found that the solid particulates as defined above provide the compositions of the present invention with improved skin feel benefits. It has been found that when such particulates are used in the skin conditioning compositions, and are formulated within the above-defined average particle diameter range, and are most typically spherical or platelet shaped, that the solid particulates provide improved cosmetic benefits to compositions. It has additionally been found that the use of such particulates in the formulation is especially useful in reducing the greasy and tacky skin feel associated with the use of a skin conditioning composition.

The solid particulates must also remain insoluble in the composition matrix, and can therefore include any inert or skin active solid particulate suitable for topical application to the hair or skin. Many of the other optional materials as described hereinabove can be selected and formulated within the composition as the solid, insoluble, particulate, provided that the formulated solid has the requisite particulate characteristics as defined herein. In this context, the term “insoluble” only means that all or most of the solid non-structuring particulates remain as solid particulates within the finished composition and are not dissolved, and also maintain the above-described average particle size, concentration, and particle morphology.

Non-limiting examples of solid particulates for use herein include inorganic powders such as gums, chalk, Fuller's earth, talc, kaolin, iron oxide, mica, sericite, muscovite, phlogopite, synthetic mica, lepidolite, inorganic pigments, biotite, lithia mica, vermiculite, magnesium carbonate, calcium carbonate, aluminum silicate, starch, smectite clays, alkyl and/or trialkyl aryl ammonium smectites, chemically modified magnesium aluminum silicate, organically modified montmorillonite clay, hydrated aluminum silicate, aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), and boron nitride; organic powder such as polyamide resin powder (nylon powder), cyclodextrin, polyethylene powder, methyl polymethacrylate powder, polystyrene powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; inorganic white pigments such as titanium dioxide, zinc oxide, and magnesium oxide. Other solid particulates fore use herein are described in U.S. Pat. No. 5,688,831 (El-Nokaly et al.) which description is incorporated herein by reference.

Preferred solid particulates for use herein are hydrophobically modified corn starch (e.g., trade name Dry-Flo from National Starch) and particulate crosslinked hydrocarbyl-substituted polysiloxane (e.g., tradename Tospearl from GE Silicone). Mixtures of the above particulates may also be used.

Other suitable solid non-structuring particulates for use herein include various moisture, sweat or sebum absorbing powders, non-limiting examples of which include silicas (or silicon dioxides), silicates, carbonates, various organic copolymers, and combinations thereof. The silicates are most typically those formed by the reaction of a carbonate or silicate with an alkali metal, alkaline earth metal, or transition metal, specific non-limiting examples of which include calcium silicate, amorphous silicas, calcium carbonate, magnesium carbonate, zinc carbonate, and combinations thereof. Non-limiting examples of some suitable silicates and carbonates for use herein are described in Van Nostrand Reinhold's Encyclopedia of Chemistry, 4^{th} edition, pages 155, 169, 556, and 849 (1984), which descriptions are incorporated herein by reference. Absorbent powders are also described in U.S. Pat. No. 6,004,584 (Peterson et al.), which description is incorporated herein by reference.

Other absorbent powders suitable for use herein include kaolin, mica, talc, starch, modified starch, microcrystalline cellulose (e.g., Avicel from FMC Corporation), or other silica-containing or non-silica-containing powder suitable for absorbing fluids from the applied surface of the body.

Preferred compositions according to the present invention are substantially free of surface active agents. By “substantially free” is meant that the composition comprises less than about 10%, preferably less than about 5%, more preferably less than about 3% surface active agents.

Deposition Efficiency:

Compositions of the present invention provide deposition of the skin conditioning or skin benefit agents onto skin with a Deposition Efficiency (DE) of at least about 30% wherein DE=[W_{after}−W₀]/[W_{before}−W₀]×100%]. Deposition Efficiency is determined using the method described below.

The test method described below is used to determine the level of deposition of skin conditioning and skin benefit agents.

Clear 3 mil thick polyethylene sheets are cut to 21.5 cm×32.0 cm. Both sides of the sheets are sprayed with ethanol, wiped with a paper towel and allowed to hang dry for a few hours. The initial substrate weight is measured using a 4-digit analytical balance and recorded as W₀.

A piece of thick, grooved vinyl shelf covering (i.e. “Groovy Easy Liner” for shelves) is clipped to a 10×13 inch plastic clipboard. The ribs are about 5 mm wide, spaced about 5 mm between ribs, and are about 1.6 mm thick with 0.55 mm thick valleys. The ribs run across the short direction of the clipboard, and serve to provide underlying texture.

One polyethylene sheet is attached to the clipboard using a clip, placing the sheet over the underlying grooved vinyl covering. 1 gram of rinse-off skin conditioning composition is applied to the sheet and spread by hand on the sheet for 30 seconds. The sheet is again weighed. This weight, prior to rinsing is recorded as W_{before}.

The sheet is rinsed for 30 seconds in warm water (100-105° F.), letting the water stream hit the top edge of the sheet and cascade down the washed area. Water flow rate is 210-230 ml/10 seconds. The sheet is hung to dry from one corner using a clothespin and dried overnight. The sheet is weighed again the next day. This weight after rinsing is recorded as W_{after}.

The deposition efficiency is calculated as: Deposition Efficiency=[W_{after}−W₀]/[W_{before}−W₀]×100%]. The preferred position efficiency is at least about 30 percent, more preferably, at least about 40 percent, most preferably, at least about 50 percent.

Skin Benefit Agent

The skin conditioning compositions of the present invention may further comprise a skin benefit agent suitable for use on the skin, and which is otherwise compatible with the other selected ingredients in the active phase of the composition. Non-limiting examples of skin benefit agents suitable for use herein are described in The CTFA Cosmetic Ingredient Handbook, Second Edition (1992), which includes a wide variety of cosmetic and pharmaceutical ingredients commonly used in the skin care industry, and which are suitable for use in the compositions of the present invention. Non-limiting examples of such skin benefit agents include abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents, skin soothing and/or healing agents. (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof. In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed. The skin benefit agents are furthered described hereinafter in details.

A) Desquamation Actives

The skin benefit agent for use herein can include desquamation actives, preferred concentrations of which range from about 0.1% to about 10%, more preferably from about 0.2% to about 5%, even more preferably from about 0.5% to about 4%, by weight of the composition. Desquamation actives enhance the skin appearance benefits of the present invention. For example, the desquamation actives tend to improve the texture of the skin (e.g., smoothness). One desquamation system that is suitable for use herein contains sulfhydryl compounds and zwitterionic surfactants and is described in U.S. Pat. No. 5,681,852, to Bissett, which description is incorporated herein by reference.

Another desquamation system that is suitable for use herein contains salicylic acid and zwitterionic surfactants and is described in U.S. Pat. No. 5,652,228 to Bissett, which description is incorporated herein by reference. Zwitterionic surfactants such as described in these applications are also useful as desquamatory agents herein, with cetyl betaine being particularly preferred.

B) Anti-Acne Actives

The skin benefit agent for use herein can also include anti-acne actives, preferred concentrations of which range from about 0.01% to about 50%, more preferably from about 1% to about 20%, by weight of the composition. Non-limiting examples of anti-acne actives suitable for use herein include resorcinol, sulfur, salicylic acid, benzoyl peroxide, erythromycin, zinc, and other similar materials.

Other non-limiting examples of suitable anti-acne actives for use herein are described in U.S. Pat. No. 5,607,980, issued to McAtee et al, which description is incorporated herein by reference.

C) Anti-Wrinkle Actives/Anti-Atrophy Actives

The skin benefit agent for use herein can also include anti-wrinkle actives or anti-atrophy actives, including sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, and skin peel agents (e.g., phenol and the like).

Hydroxy acids as skin benefit agents herein include salicylic acid and salicylic acid derivatives, preferred concentrations of which range from about 0.01% to about 50%, more preferably from about 0.1% to about 10%, even more preferably from about 0.5% to about 2%, by weight of the composition.

Other non-limiting examples of suitable anti-wrinkle actives for use herein are described in U.S. Pat. No. 6,217,888, issued to Oblong et al, which description is incorporated herein by reference.

D) Anti-Oxidants/Radical Scavengers

The skin benefit agent for use herein can also include anti-oxidants or radical scavengers, preferred concentrations of which range from about 0.1% to about 10%, more preferably from about 1% to about 5%, by weight of the composition.

Non-limiting examples of anti-oxidants or radical scavengers for use herein include ascorbic acid and its salts, ascorbyl esters of fatty acids, ascorbic acid derivatives (e.g., magnesium ascorbyl phosphate, sodium ascorbyl phosphate, ascorbyl sorbate), tocopherol, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

E) Chelators

The skin benefit agent for use herein can also include chelating agents. As used herein, the term “chelating agent” or “chelator” refers to those skin benefit agents capable of removing a metal ion from a system by forming a complex so that the metal ion cannot readily participate in or catalyze chemical reactions.

The chelating agents as skin benefit agents for use herein are preferably formulated at concentrations ranging from about 0.1% to about 10%, more preferably from about 1% to about 5%, by weight of the composition. Non-limiting examples of suitable chelating agents are described in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995, which descriptions are incorporated herein by reference.

Preferred chelating agents for use in the active phase of the compositions of the present invention include furildioxime, furilmonoxime, and derivatives thereof.

F) Flavonoids

The skin benefit agent for use herein includes flavonoid compounds suitable for use on the hair or skin, preferred concentrations of which range from about 0.01% to about 20%, more preferably from about 0.1% to about 10%, more preferably from about 0.5% to about 5%, by weight of the composition.

Non-limiting examples of flavonoids compounds suitable for use as skin benefit agents include flavanones such as unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents.

Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

Among these flavanoid compounds, preferred are unsubstituted flavanone, methoxy flavanones, unsubstituted chalcone, 2′,4-dihydroxy chalcone, isoflavone, flavone, and mixtures thereof, more preferably soy isoflavones.

Other non-limiting examples of flavanoid compounds suitable for use as skin benefit agents herein are described in U.S. Pat. Nos. 5,686,082 and 5,686,367, which descriptions are incorporated herein by reference.

G) Anti-Inflammatory Agents

The skin benefit agent for use in the active phase of the composition can include anti-inflammatory agents, preferred concentrations of which range from about 0.1% to about 10%, more preferably from about 0.5% to about 5%, by weight of the composition.

Non-limiting examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Nonsteroidal anti-inflammatory agents are also suitable for use herein as skin benefit agents in the active phase of the compositions. Non-limiting examples of non-steroidal anti-inflammatory agents suitable for use herein include oxicams (e.g., piroxicam, isoxicam, tenoxicam, sudoxicam, CP-14,304); salicylates (e.g., aspirin, disalcid, benorylate, trilisate, safapryn, solprin, diflunisal, fendosal); acetic acid derivatives (e.g., diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac); fenamates (e.g., mefenamic, meclofenamic, flufenamic, niflumic, tolfenamic acids); propionic acid derivatives (e,g., ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic); pyrazoles (e.g., phenylbutazone, oxyphenbutazone, feprazone, azapropazone, trimethazone); and combinations thereof as well as any dermatologically acceptable salts or esters of thereof.

Other non-limiting examples of suitable anti-inflammatory or similar other skin benefit agents include candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, sea whip extract, and combinations thereof.

Other non-limiting examples of suitable anti-inflammatory or similar other skin benefit agents include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific non-limiting examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, and combinations thereof.

H) Anti-Cellulite Agents

The skin benefit agent for use in the active phase of the compositions of the present invention anti-cellulite agents, non-limiting examples of which include xanthine compounds such as caffeine, theophylline, theobromine, aminophylline, and combinations thereof.

I) Topical Anesthetics

The skin benefit agent for use in the active phase of the compositions of the present invention include topical anesthetics, non-limiting examples of which include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, pharmaceutically acceptable salts thereof, and combinations thereof.

J) Tanning Actives

The skin benefit agent for use in the active phase of the compositions of the present invention include tanning actives, preferred concentrations of which range from about 0.1% to about 20% by weight of the composition. Non-limiting examples of such tanning agents include dihydroxyacetone, which is also known as DHA or 1,3-dihydroxy-2-propanone.

K) Skin Lightening Agents

The skin benefit agent for use in the active phase of the compositions of the present invention can include skin lightening agents, preferred concentrations of which range from about 0.1% to about 10%, more preferably from about 0.2% to about 5%, more preferably from about 0.5% to about 2%, by weight of the composition. Non-limiting examples of skin lightening agents suitable for use herein include kojic acid, arbutin, ascorbic acid and derivatives thereof (e.g., magnesium ascorbyl phosphate or sodium ascorbyl phosphate), and extracts (e.g., mulberry extract, placental extract). Non-limiting examples of skin lightening agents suitable for use herein also include those described in WO 95/34280, WO 95/07432, and WO 95/23780.

L) Skin Soothing and Skin Healing Actives

The skin benefit agent for use in the active phase of the compositions of the present invention include skin soothing and skin healing actives, preferred concentrations of which range from about 0.1% to about 30%, more preferably from about 0.5% to about 20%, still more preferably from about 0.5% to about 10%, by weight of the composition. Non-limiting examples of skin soothing or skin healing actives suitable for use herein include panthenoic acid derivatives (e.g., panthenol, dexpanthenol, ethyl panthenol), aloe vera, allantoin, bisabolol, and dipotassium glycyrrhizinate.

M) Antimicrobial Actives

The skin benefit agent for use in the active phase of the compositions of the present invention includes antimicrobial actives, preferred concentrations of which range from about 0.001% to about 10%, more preferably from about 0.01% to about 5%, and still more preferably from about 0.05% to about 2%, by weight of the compositions.

Non-limiting examples of antimicrobial actives for use herein includes β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, clotrimazole, and combinations thereof.

N) Sunscreen Actives

The skin benefit agent for use in the active phase of the compositions of the present invention may comprise a sunscreen active, either organic or inorganic suncscreen actives. Among the inorganic sunscreens useful hererin are metallic oxides such as titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof.

The concentration of the sunscreen active for use in the composition preferably ranges from about 0.1% to about 20%, more typically from about 0.5% to about 10%, by weight of the composition. Exact amounts of such sunscreen actives will vary depending upon the sunscreen or sunscreens chosen and the desired Sun Protection Factor (SPF).

A wide variety of conventional organic sunscreen actives are also suitable for use herein, non-limiting examples of which include p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane. Among these sunscreens, preferred are 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethylaminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and combinations thereof.

Non-limiting examples of other sunscreen actives suitable for use herein include those described in U.S. Pat. No. 4,937,370 issued to Sabatelli on Jun. 26, 1990, and U.S. Pat. No. 4,999,186 issued to Sabatelli & Spirnak on Mar. 12, 1991, which descriptions are incorporated herein by reference. Among those sunscreen actives described, preferred are 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof. Especially preferred sunscreen actives include 4,4′-t-butylmethoxydibenzoylmethane, 2-ethylhexyl-p-methoxycinnamate, phenyl benzimidazole sulfonic acid, and octocrylene.

Optional Ingredients

The skin conditioning compositions of the present invention may further comprise other optional ingredients that may modify the physical, chemical, cosmetic or aesthetic characteristics of the compositions or serve as additional “active” components when deposited on the skin. The compositions may also further comprise optional inert ingredients. Many such optional ingredients are known for use in personal care compositions, and may also be used in the skin conditioning compositions herein, provided that such optional materials are compatible with the essential materials described herein, or do not otherwise unduly impair product performance.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. These optional materials can be used in any aspect of the compositions of the present invention, including either of the active or cleansing phases as described herein.

Other optional ingredients include silicone elastomer powders and fluids to provide any of a variety of product benefits, including improved product stability, application cosmetics, emolliency, conditioning, and so forth. The concentration of the silicone elastomers in the composition preferably ranges from about 0.1% to about 20%, more preferably from about 0.5% to about 10%, by weight of the composition. In this context, the weight percentages are based upon the weight of the silicone elastomers material itself, excluding any silicone-containing fluid that typically accompanies such silicone elastomers materials in the formulation process. The silicone elastomers suitable for optional use herein include emulsifying and non-emulsifying silicone elastomers, non-limiting examples of which are described in U.S. Ser. No. 09/613,266 (assigned to The Procter & Gamble Company), which description is incorporated herein by reference.

### Method of Use

The skin conditioning compositions of the present invention are preferably applied topically to the desired area of the hair or skin in an amount sufficient to provide effective delivery of the skin benefit agent to the applied surface, or to otherwise provide effective skin conditioning benefits. The compositions are preferably diluted with water during, or after topical application, and then subsequently rinsed or wiped off of the applied surface, preferably rinsed off of the applied surface using water or a water-insoluble substrate in combination with water.

The present invention is also directed to methods of using the skin conditioning compositions of the present invention, wherein the skin conditioning compositions are applied to the skin of the consumer while showering then rinsed or wiped off.

The present invention is therefore also directed to methods of providing effective delivery of the desired skin benefit agent, and the resulting benefits from such effective delivery as described herein, to the applied surface through the above-described application of the compositions of the present invention.

### Method of Manufacture

The skin conditioning compositions of the present invention may be prepared by any known or otherwise effective technique, suitable for making and formulating the desired product form. Specific non-limiting examples of such methods as they are applied to specific embodiments of the present invention are described in the following examples.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, i.e., wt/wt percentages, unless otherwise specified.

Each of the exemplified compositions provides improved cosmetics during and after application, including reduced greasy or sticky skin feel, and provides improved deposition or effectiveness of the skin benefit agent deliver from each prepared composition.

### Examples 1-4

The following examples described in Table 1 are non-limiting examples of skin conditioning compositions with solid particulates of the present invention.

TABLE I
Rinse-off Skin Conditioning Compositions

| | Example 1 | Example 2 | Example 3 | Example 4 |
|---|---|---|---|---|
| Ingredient | wt % | wt % | wt % | Wt % |
| Petrolatum | 39.9 | 39.9 | 44.9 | 39.9 |
| Mineral Oil | 39.9 | 39.9 | 44.9 | — |
| Sunflower Seed Oil | — | — | — | 39.9 |
| Tospearl 2000 (from GE) | — | 20 | — | — |
| Dry-Flo AF (from | 20 | — | 10 | 20 |
| National Starch) | | | | |
| Perfume | 0.2 | 0.2 | 0.2 | 0.2 |

The compositions described above are prepared by conventional formulation and mixing techniques. The skin conditioning compositions are prepared by adding petrolatum into a mixing vessel. Heat the vessel to 140° F. Then, add mineral oil or sunflower seed oil and Dry-Flo AF or Tospearl with agitation. Let the vessel cool down with slow agitation and add perfume when the temperature drops below 100° F.

These compositions are used as a rinse-off skin conditioner to deliver conditioning benefits to the skin. The present compositions have excellent skin conditioning benefits with acceptable aesthetics profile.

### Examples 5-8

The following examples described in Table 2 are non-limiting examples of skin conditioning compositions of the present invention.

TABLE 1
Rinse-off Skin Conditioning Compositions with Actives

| | Example 5 | Example 6 | Example 7 | Example 8 |
|---|---|---|---|---|
| Ingredient | wt % | wt % | wt % | wt % |
| Petrolatum | 34.9 | 34.9 | 34.9 | 34.9 |
| Mineral Oil | 34.9 | 34.9 | 34.9 | 34.9 |
| Tocopherol Nicotinate | 10 | — | — | 5 |
| Niacinamide | — | 10 | — | — |
| Farnesol | — | — | 10 | 5 |
| Dry-Flo AF | 20 | 20 | 20 | 20 |
| Perfume | 0.2 | 0.2 | 0.2 | 0.2 |

The compositions described above are prepared by conventional formulation and mixing techniques. The skin conditioning compositions are prepared by adding petrolatum into a mixing vessel. Heat the vessel to 140° F. Then, add mineral oil and Dry-Flo AF, and skin actives (Tocopherol Nicotinate, Niacinamide, Farnesol) with agitation. Let the vessel cool down with slow agitation and add perfume when the temperature drops below 100° F.

These compositions are used as rinse-off skin conditioner to deposit anti-aging active onto the skin. The present compositions have excellent skin conditioning benefits with acceptable aesthetics profile.

What is claimed is:

1. A skin conditioning composition comprising:
(i) a lipophilic carrier; and
(ii) solid particulates;
wherein the composition has a Deposition Efficiency (DE) of at least about 30% wherein DE=[W_{after}−W₀]/[W_{before}−W₀]×100%].

2. A skin conditioning composition comprising:
(i) at least about 10% by weight of a lipophilic carrier; and
(ii) from about 1.0% to about 90% by weight of solid particulates;
wherein the composition has a Deposition Efficiency (DE) of at least about 30% wherein DE=[W_{after}−W₀]/[W_{before}−W₀]×100%].

3. A skin conditioning composition according to claim 1, wherein the lipophilic carrier has a Vaughn Solubility Parameter of from about 5 to about 10.

4. A skin conditioning composition according to claim 1, wherein the lipophilic carrier has a Consistency value of from about 1 poise to about 2,000 poise.

5. A skin conditioning composition according to claim 1, wherein the lipophilic carrier has a Shear Index of from about 0.1 to about 0.8.

6. A skin conditioning composition according to claim 1, wherein at least 10% by weight of the lipophilic carrier is selected from the group consisting of petrolatum, mineral oil micro-crystalline waxes, paraffins, ozokerite, polyethylene, polybutene, polydecene and perhydrosqualene dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and combinations thereof.

7. A skin conditioning composition according to claim 1, wherein the solid particulate is selected from the group consisting of hydrophobically modified corn starch, particulate crosslinked hydrocarbyl-substituted polysiloxane, chalk, Fuller's earth, talc, kaolin, iron oxide, mica, sericite, muscovite, phlogopite, synthetic mica, lepidolite, inorganic pigments, biotite, lithia mica, vermiculite, magnesium carbonate, calcium carbonate, aluminum silicate, starch, smectite clays, alkyl and/or trialkyl aryl ammonium smectites, chemically modified magnesium aluminum silicate, organically modified montmorillonite clay, hydrated aluminum silicate, aluminum starch octenyl succinate barium silicate, calcium silicate, magnesium silicate, strontium silicate, metal tungstate, magnesium, silica alumina, zeolite, barium sulfate, calcined calcium sulfate, calcium phosphate, fluorine apatite, hydroxyapatite, ceramic powder, metallic soap, boron nitride, organic powder, cyclodextrin, polyethylene powder, methyl polymethacrylate powder, polystyrene powder, copolymer powder of styrene and acrylic acid, benzoguanamine resin powder, poly(ethylene tetrafluoride) powder, carboxyvinyl polymer, hydroxyethyl cellulose, sodium carboxymethyl cellulose, ethylene glycol monostearate, titanium dioxide, zinc oxide, magnesium oxide and mixtures thereof.

8. A skin conditioning composition according to claim 1, wherein the composition is substantially free of surface active agents.

9. A skin conditioning composition according to claim 1, wherein the solid particulates are non-structuring.

10. A skin conditioning composition according to claim 1, wherein the solid particulates have an average particle diameter of from about 1 μm to about 100 μm

11. A skin conditioning composition according to claim 1, further comprising a skin benefit agent.

12. A skin conditioning composition according to claim 11, wherein the skin benefit agent is selected from the group consisting of desquamation actives, anti-acne actives, anti-wrinkle and anti-atrophy actives, anti-oxidants and radical scavengers, chelators, flavonoids, anti-inflammatory agents, anti-cellulite agents, topical anesthetics, tanning actives, skin lightening agents, skin soothing and skin healing actives, antimicrobial actives, suncreens, and combinations thereof.

13. A method of conditioning the skin comprising the steps of applying a skin conditioning composition comprising:
(i) a lipophilic carrier; and
(ii) solid particulates;
wherein the composition has a Deposition Efficiency (DE) of at least about 30% wherein DE=[W_{after}−W₀]/[W_{before}−W₀]×100%] and, removing the composition once applied by means selected from by rinsing, wiping and mixtures thereof.

14. A method of conditioning the skin comprising the steps of applying a skin conditioning composition comprising:
(i) at least about 10% by weight of a lipophilic carrier; and
(ii) from about 1.0% to about 90% by weight of solid particulates;
wherein the composition has a Deposition Efficiency (DE) of at least about 30% wherein DE=[W_{after}−W₀]/[W_{before}−W₀]×100%] and removing the composition once applied by means selected from by rinsing, wiping and mixtures thereof.

15. A method of conditioning the skin according to claim 13, wherein the lipophilic carrier has a Vaughn Solubility Parameter of from about 5 to about 10.

16. A method of conditioning the skin according to claim 13, wherein the lipophilic carrier has a Consistency value of from about 1 poise to about 2,000 poise.

17. A method of conditioning the skin according to claim 13, wherein the lipophilic carrier has a Shear Index of from about 0.1 to about 0.8.

18. A method of conditioning the skin according to claim 13, wherein at least 10% by weight of the lipophilic carrier is selected from the group consisting of petrolatum, mineral oil micro-crystalline waxes, paraffins, ozokerite, polyethylene, polybutene, polydecene and perhydrosqualene. dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and combinations thereof.

19. A method of conditioning the skin according to claim 13, wherein the solid particulate is selected from the group consisting of hydrophobically modified corn starch, particulate crosslinked hydrocarbyl-substituted polysiloxane, chalk, Fuller's earth, talc, kaolin, iron oxide, mica, sericite, muscovite, phlogopite, synthetic mica, lepidolite, inorganic pigments, biotite, lithia mica, vermiculite, magnesium carbonate, calcium carbonate, aluminum silicate, starch, smectite clays, alkyl and/or trialkyl aryl ammonium smectites, chemically modified magnesium aluminum silicate, organically modified montmorillonite clay, hydrated aluminum silicate, aluminum starch octenyl succinate barium silicate, calcium silicate, magnesium silicate, strontium silicate, metal tungstate, magnesium, silica alumina, zeolite, barium sulfate, calcined calcium sulfate, calcium phosphate, fluorine apatite, hydroxyapatite, ceramic powder, metallic soap, boron nitride, organic powder, cyclodextrin, polyethylene powder, methyl polymethacrylate powder, polystyrene powder, copolymer powder of styrene and acrylic acid, benzoguanamine resin powder, poly(ethylene tetrafluoride) powder, carboxyvinyl polymer, hydroxyethyl cellulose, sodium carboxymethyl cellulose, ethylene glycol monostearate, titanium dioxide, zinc oxide, magnesium oxide and mixtures thereof.

20. A method of conditioning the skin according to claim 13, wherein the solid particulates are non-structuring.

21. A method of conditioning the skin according to claim 13, wherein the solid particulates have an average particle diameter of from about 1 μm to about 100 μm

22. A method of conditioning the skin according to claim 13, further comprising a skin benefit agent.

23. A method of conditioning the skin according to claim 22, wherein the skin benefit agent is selected from the group consisting of desquamation actives, anti-acne actives, anti-wrinkle and anti-atrophy actives, anti-oxidants and radical scavengers, chelators, flavonoids, anti-inflammatory agents, anti-cellulite agents, topical anesthetics, tanning actives, skin lightening agents, skin soothing and skin healing actives, antimicrobial actives, sunscreens, and combinations thereof.

24. A method of conditioning the skin according to claim 13 wherein the composition is substantially free of surface active agents.
